(19) United States
(12) Patent Application Publication
(10) Pub. No.: US 2005/0148979 A1
(43) Pub. Date: Jul. 7, 2005

(54) Packaging component with sensory cue for opening

(76) Inventors: Joseph Di Palma, Neenah, WI (US); Adrienne R. Loyd, Neenah, WI (US); Marie A. Mathews, Appleton, WI (US); Kristi J. Bryant, Appleton, WI (US)

Correspondence Address: BRINKS HOFER GILSON & LIONE, P.O. BOX 10395, CHICAGO, IL, 60610, US

(21) Appl. No.: 10/750,424

(22) Filed: Dec. 30, 2003

(51) Int. Cl. A61F 13/15; A61F 13/20

(52) U.S. Cl. 604/385.02

(57) ABSTRACT

A personal care product includes a personal care product component disposed in a packaging component. The packaging component includes a panel and a flap having a free edge positioned adjacent the panel. One of the flap and panel includes a sensory cue directed at the free edge of the flap. The sensory cue provides indicia as to the location of the free edge and terminates at the free edge. In one embodiment, the packaging component includes a front panel, a back panel and a flap overlying a portion of the front panel. In one embodiment, the sensory cue is configured as a visual cue. In another aspect, a method of using a personal care product includes locating the free edge of the packaging component using the visual cue and grasping the flap and opening the packaging component to access the personal care product component.

### BACKGROUND

The present invention relates generally to a personal care product, and in particular, to a packaging component for a personal care product that includes a sensory cue directed at a portion of the packaging component so as to aid the user in opening the packaging component.

Often, personal care products, including for example feminine pads and panty liners, are individually wrapped in a pouch or similar package, which includes a flap that closes the pouch. Typically, such pouches are a solid color, or have a pattern that covers the entirety of the package. As such, it can be difficult for the user to locate the flap to open the pouch, as the flap is not visually distinguishable from the remainder of the package. The problem of locating the flap can be exacerbated for users with impaired eyesight.

### SUMMARY

Briefly stated, in one aspect, a personal care product includes a personal care product component disposed in a packaging component. The packaging component includes a panel and a flap having a free edge positioned adjacent the panel. At least one of the flap and panel includes a sensory cue directed at the free edge of the flap. The sensory cue provides indicia as to the location of the free edge and terminates at the free edge. In one embodiment, the packaging component includes a front panel, a back panel and a flap overlying a portion of the front panel. In one embodiment, both of the front panel and flap have a visual cue.

In one embodiment, the sensory cue is configured as a visual cue. In various exemplary embodiments, the visual cue can include color and/or a pattern, formed for example by printing or embossing or texturing portions of the pouch. The other of the front panel and flap may have a color and/or pattern that is different than the visual cue. In other embodiments, the sensory cue is configured as a tactile cue. Of course, the cue can include both visual and tactile aspects.

In another aspect, a method of using a personal care product includes locating the free edge of the packaging component using the sensory cue and grasping the flap and opening the packaging component to access the personal care product component.

In another aspect, the packaging component has a first length and the flap and panel have a sensory cue disposed across the free edge of the flap. The sensory cue has a second length less than about 33% of the first length.

The various aspects provide significant advantages over other personal care products. For example and without limitation, in the first aspect, the sensory cue draws attention to the free edge of the flap so as to facilitate the opening thereof by the end user. In addition, the method of opening the packaging component is made intuitive by providing a sensory cue.

The foregoing paragraphs have been provided by way of general introduction, and are not intended to limit the scope of the following claims. The presently preferred embodiments, together with further advantages, will be best understood by reference to the following detailed description taken in conjunction with the accompanying drawings.

### BRIEF DESCRIPTION OF THE DRAWINGS

FIG. 1 is a perspective view of a packaging component in an open configuration with a personal care product component in a partially unfolded configuration.

FIG. 2 is a body-side plan view of an exemplary personal care product component with a portion thereof partially cut away.

FIGS. 3A-3F are plan views of various packaging components.

FIG. 4 is a schematic cross-section of one embodiment of a packaging component, with various dimensions and proportions at the ends thereof exaggerated for the sake of illustration.

FIG. 5A is a plan view of one embodiment of a packaging component material in an unfolded configuration.

FIG. 5B is a plan view of the packaging component material shown in FIG. 5A in a folded configuration.

FIG. 6 is a plan view of an alternative embodiment of a packaging component.

FIG. 7 is a plan view of an alternative embodiment of a packaging component.

FIG. 8 is schematic graphical illustration of hue, luminosity and saturation/vividness.

### DETAILED DESCRIPTION OF THE PRESENTLY PREFERRED EMBODIMENTS

It should be understood that the term “personal care product” as used herein refers to any article used to control bodily fluids, and includes “absorbent products,” which refers to any article configured to absorb and retain bodily exudates, including urine, bowel movements, blood and menses, and includes such a product in a packaged and unpackaged configuration. As such, personal care products, as used herein, includes without limitation diapers, pull-up garments, adult incontinence garments, male incontinence products, tampons, vaginal suppositories, panty liners, pads, sanitary napkins, tissues, wipes, etc. For example, personal care products include without limitation Poise® feminine care products, including pantiliners and pads, and Kotex® feminine care products, including pads, tampons and liners, all available from Kimberly-Clark Corporation, Neenah, Wis. Various exemplary products are disclosed in U.S. Pat. No. 6,315,765, entitled “Elasticized Absorbent Pad,” and U.S. patent application Ser. No. 10/392,116, filed Mar. 19, 2003 and entitled “Multilayer Absorbent Article”, the entire disclosures of which are hereby incorporated herein by reference.

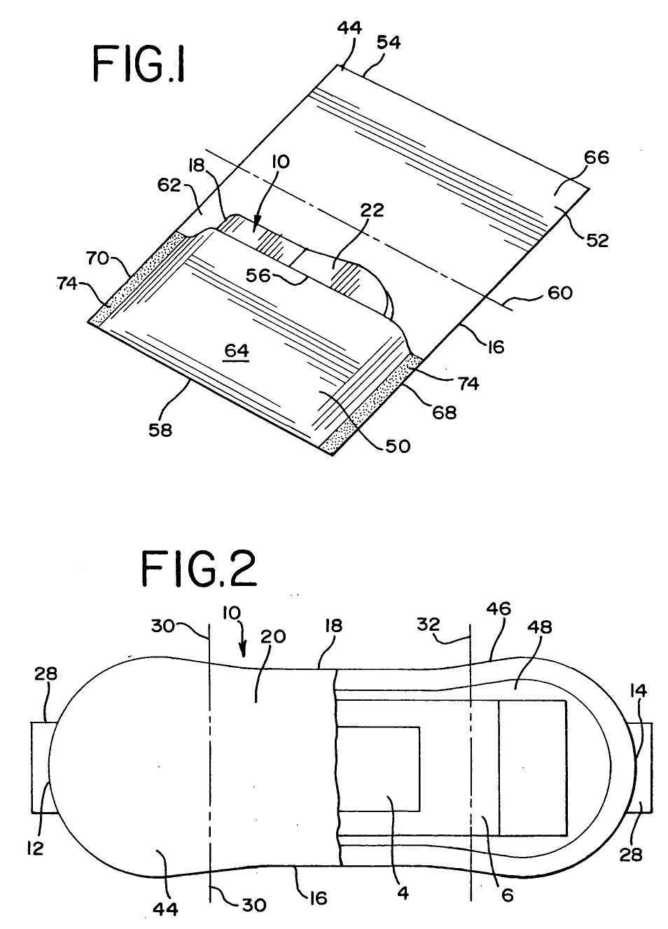

Referring to FIGS. 1 and 2, an exemplary absorbent product 10 is shown as including an outer cover 46 (otherwise referred to as a baffle), an absorbent core 48, a tissue layer 6, a surge layer 4 and a body side liner 44. Preferably, the body-side liner 44 is liquid-permeable. The absorbent product 10 also has a first side and a second side 18. The first and second sides 16, 18, respectively, are the longitudinal sides of the elongated absorbent product. The sides can be contoured, for example in a concave shape, or they can be linear. The sides can further include flaps (not shown) that extend laterally outward. In one embodiment (not shown), one or more elastic elements are disposed along the sides to form a gasket with the body of the user. In one embodiment, the elastic elements are disposed between the liner and the outer cover.

The absorbent product 10 has a first body side surface 20 and a second garment side surface 22. Applied to at least a portion of the second garment side surface 22 is a garment attachment adhesive. In various embodiments, the garment attachment adhesive is configured as a single band of adhesive or as two or more spaced apart strips. Alternatively, the garment attachment adhesive includes a swirl pattern of adhesive which encompasses a major portion of the second garment surface 22 of the absorbent article 10.

A release strip 28, also known as a releasable peel strip, is removably secured to the garment attachment adhesive and serves to prevent premature contamination of the adhesive before the absorbent article 10 is secured to the crotch portion of an undergarment. In various embodiments, the garment attachment adhesive is designed to be secured to the inner crotch portion of an undergarment so as to keep the absorbent product in register with the body of the user. The release strip 28 may extend beyond one or both of the ends 12, 14 of the outer cover, as shown in FIG. 2, or it may have a lesser length than the outer cover, as shown in FIG. 1.

The body side liner 44, which is preferably liquid permeable, is formed from one or more of a nonwoven material such as spunbond or a perforated thermoplastic film. The term “non-woven” web or material, as used herein, means a web having a structure of individual fibers or filaments that are interlaid, but not in an identifiable manner and without the aid of textile weaving or knitting, as in a knitted or woven fabric. The baffle 46 should be liquid-impermeable and can be formed out of a thin layer of thermoplastic film, such as polyethylene. The liquid permeable liner 44 and the liquid-impermeable baffle 46 are peripherally sealed together to enclose the absorbent core 48 to form the absorbent article 10. Alternatively, the liner 44 can be wrapped around both the absorbent 48 and the baffle 46 to form a wrapped pad. The liner 44 and baffle 46, and other components of the absorbent product, can be joined for example with adhesive bonds, sonic bonds, thermal bonds, pinning, stitching or any other attachment techniques known in the art, as well as combinations thereof.

The absorbent core 48 is designed to absorb body exudates, including menstrual fluid, blood, urine, and other body fluids. The absorbent core 48 can consist of one or more layers of absorbent material. The layers can consist of similar materials or different materials. Suitable materials for the absorbent core 48 include cellulose, wood pulp fluff, rayon, cotton, and meltblown polymers such as polyester, polypropylene or coform. Coform is a meltblown air-formed combination of meltblown polymers, such as polypropylene, and absorbent staple fibers, such as cellulose. A preferred material is wood pulp fluff, for it is low in cost, relatively easy to form, and has great absorbency.

The absorbent core 48 can also be formed from a composite comprised of a hydrophilic material that can be formed from various natural or synthetic fibers, wood pulp fibers, regenerated cellulose or cotton fibers, or a blend of pulp and other fibers. A preferred material is an airlaid tissue.

In one embodiment, the absorbent core also includes a superabsorbent material, which increases the ability of the absorbent core to absorb a large amount of fluid in relation to its own weight. Typical superabsorbents used in absorbent articles such as sanitary napkins, can absorb anywhere from 5 to 60 times their weight in body fluid. The superabsorbent materials can be inserted as particles or in sheet form. Hydroxyfunctional polymers have been found to be good superabsorbents for sanitary napkins. Such superabsorbents are commercially available from Dow Chemical, Hoechst-Celanese, and Stockhausen, Incorporated, among others, and are a partially neutralized salt of cross-linked copolymer of polyacrylic acid and polyvinyl alcohol having an absorbency under load value above 25. Other types of superabsorbent materials known to those skilled in the art can also be used.

Additional layers or substrates, including for example, the liquid acquisition and distribution layer 5, also referred to as a surge or transfer layer, and a tissue layer 6 are also incorporated into the absorbent product, for example between the liner 44 and the absorbent core 48. In one embodiment, the transfer layer is shorter than the absorbent core. In one embodiment, the absorbent core, transfer layer and other components, such as tissue layers, are free floating (unattached) between the outer cover and the liner, which are secured along only the peripheral edges thereof. Alternatively, the absorbent core, transfer layer and other components are attached to one or both of the outer cover and liner and/or to each other.

Referring to FIGS. 1 and 2, the absorbent product 10 is shown in a folded configuration. For example, the absorbent product can be folded along a pair of fold lines 30, 32 to form a tri-fold configuration. In other embodiments, the absorbent product can be bi-folded, flat or rolled. The absorbent product is then inserted into an individual packaging component, otherwise referred to as a wrapper or pouch. Alternatively, a plurality (meaning two or more) of products can be disposed in a single pouch, and a plurality of pouches can be disposed in a package. One product/packaging configuration is shown in U.S. Pat. No. 6,601,706, which is hereby incorporated by reference. The products can be oriented in various ways within the individual packaging component, for example with the fold lines 30, 32 running parallel or perpendicular to the sides of the packaging component 68, 70.

In one embodiment, the packaging component is formed as a portion of the product component. For example, an outer cover, such as the baffle material, can form a packaging component, as shown for example in U.S. Pat. No. 5,993,430, which is hereby incorporated herein by reference. It should be understood that in these embodiments, the product component is considered to be disposed in the packaging component when the packaging element is sealed or otherwise closed. In other embodiments, the product component is rolled, with a flap portion of a packaging component overlying a panel portion of the packaging component, regardless of whether the packaging component is integral with the product component or separate therefrom.

Referring to FIGS. 1, 3A-3F and 4, the pouch 50 is formed from a strip or web 52 of material having a first and second ends having free edge 54, 56. It should be understood that the term “free edge” refers to an edge that is unattached after the package component is opened, regardless of whether the free edge is attached when the package component is closed. Accordingly, one or both of the free edges may be formed along a perforation line, or may be adhered to an underlying layer, with the edge defined by the perforation line being a “free edge” after the perforation line is broken. The free edge can be a single layer cut or formed edge, or can include a double-layer folded edge, or can include an edge formed by a plurality of layers. The pouch material can be formed from a non-woven material, films, paper, laminates, and/or cloth (including woven) materials, and combinations thereof. For example, the pouch can be made as disclosed in U.S. patent application Ser. No. 10/022,808, filed Dec. 18, 2001, the entire disclosure of which is hereby incorporated herein by reference. In one embodiment, the pouch is made of a film/spunbond laminate material available from Kimberly-Clark Corp, and know as HBSTL (“highly breathable stretch thermal laminate”), and which material is further disclosed in U.S. Pat. No. 6,276,032, the entire disclosure of which is hereby incorporated herein by reference.

Each of the first and second ends are folded along fold lines 58, 60 that define the top and bottom edge of the pouch respectively. The folded pouch has a back 62, a front 64 and a flap 66. The front and back 64, 62 are secured along side edges 68, 70 thereof to form a pocket shaped to receive the absorbent product. In one embodiment, the pocket and pouch are shaped and dimensioned to receive a single product component, which is individually wrapped in the pouch. The flap 66 is folded over the front 64 such that the free edge 54 of the flap overlies the front 64. The front 64 has a covered or overlap portion 57 extending between the free edge 54 (exterior) and the free edge 56 (interior), which covered or overlapped portion 57 underlies the flap.

In one embodiment, there is no overlap. Rather, the free edges 54, 56 abut. In this embodiment, the flap is defined merely as another panel.

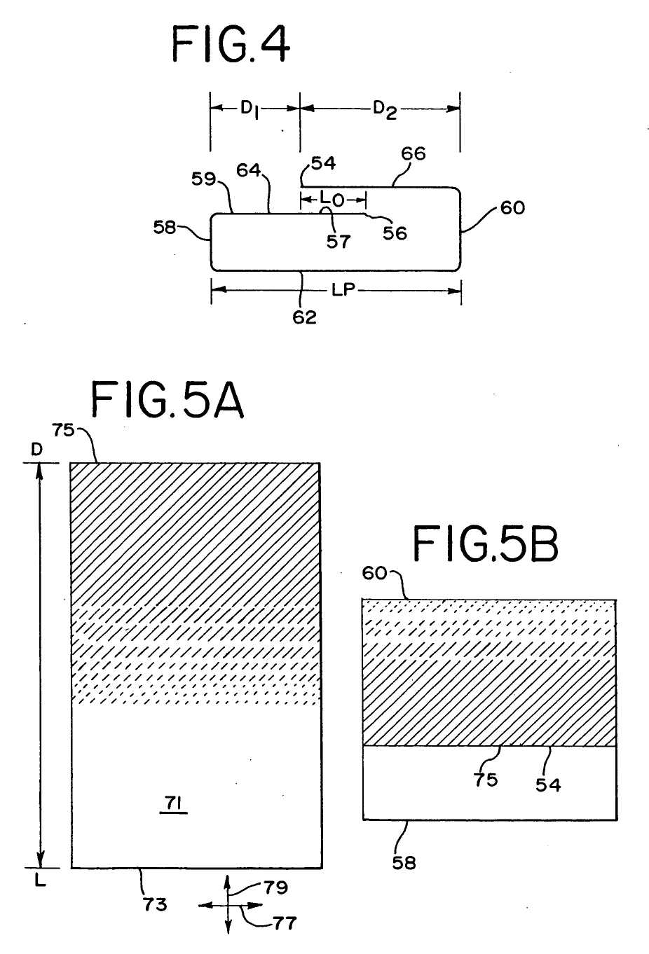

In one example, the portion 57 has a length (Lo) of about 5 mm between the edges 54, 56. In various embodiments, the overlap distance (Lo) is less than or equal to about 95% of the overall packaging component length (L) in a closed configuration (shown in FIG. 4), more desirable less than or equal to about 35% of the packaging component length, and more desirably less than or equal to about 20% of the packaging component length. In various embodiments, the free edge 54 is positioned a distance (D1, D2) from either edge 58, 60 that is greater than or equal to about 10% of the overall length (Lp) of the packaging component (in a closed configuration), more desirably greater than or equal to about 30%, and more desirably about 50% of the packaging component length. The front further includes an uncovered second portion 59 extending between the free edge 54 and the bottom edge 58. Of course, it should be understood that the length and width of the product and packaging components can vary according to the type of product and the size of the product.

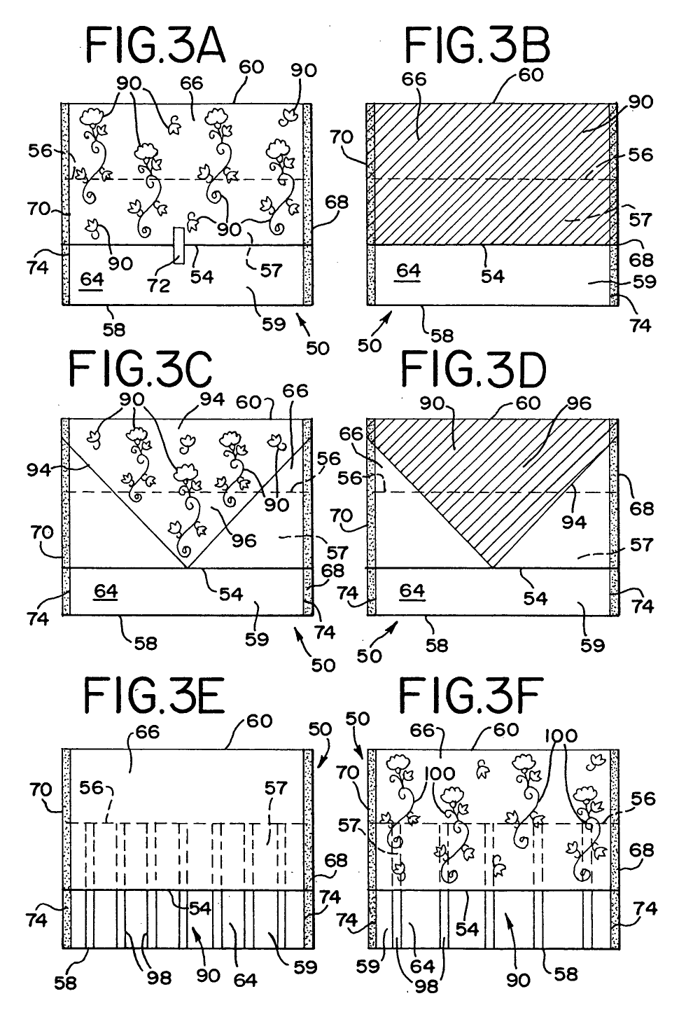

In one embodiment, the flap 66 is releasably secured to the front 64. For example, a fastening element 72, shown as a tab in FIG. 3A, is secured across the free edge 54 of the flap 66 to secure the flap 66 to the front 64. The fastening element can be releasably secured to both of the flap and front, or it can be fixedly secured to one of the flap and front and releasably secured to the other thereof. The fastening element can be formed as adhesive (e.g., tape), a snap, a button, a mechanical fastener (e.g., hook and loop), a tie, or as any other device known by those skilled in the art. The adhesive can be applied as a ribbon, dot or swirl pattern. The fastening element can have various alternative shapes, including by not limited to a square, rectangle, triangle, circle, oval, obround, oblong or diamond shape, or any other irregular shape or pattern. In an alternative embodiment, the fastening element is formed on the inside of the flap such that it engages the front as the flap is folded thereover and is not visible to the user. In yet another alternative embodiment, the flap is simply sealed to the front with a heat seal or other weld, with the weld defining the fastening element. In another embodiment, the flap is not sealed or otherwise attached to the front, but rather is simply folded thereover. Alternatively, the sides of the flap are sealed to the back and to the front, with the side seals being breakable in response to a user grasping and lifting the flap.

In some embodiments, the flap 66 is refastenably secured to the front 64, while in others, the flap is not intended to be secured to the front once the packaging component is opened. For example, in one embodiment, the free edge 54 is defined by a perforation line, with the flap not being refastenable after the perforation is broken.

A pair of side seals 74 secures the front 64 to the back 62, and the flap 66 to the back 62 and to the front 64. The side seals are desirably formed after the front is folded over the back and the flap is folded over the back and the front, although the front could first be sealed to the back, and the flap then sealed to one or both of the back and front. In an alternative embodiment, the flap is not sealed at all along the sides thereof, or anywhere else. In one embodiment, the side seals 74 are frangible, meaning they can be easily broken such that the flap 66 can be separated from the front 64 and back 62, and such that the front 64 can be easily separated from the back 62, wherein the product component 10 is exposed for removal from the pouch by the user.

Referring to FIGS. 3A-3F, at least one of the flap 66 and the front 64 includes a sensory cue 90, shown as a visual cue, that is directed at the free edge 54. The visual cue 90 desirably terminates at the free edge 54 and provides indicia as to the location of the free edge. The terms “directed at” and “terminates” do not require that the visual cue necessarily contact or run all of the way to the free edge 54, as shown for example in FIGS. 3A and 3C, wherein a pattern 92 is applied over portions of the flap 66 but with a lowermost portion thereof being slightly spaced from the edge 54. The visual cue 90 can take many forms, and can include one or more colors, shapes, graphics, text, alpha-numeric characters, and/or patterns, including indicia formed by dying, printing and/or embossing, or by otherwise altering the relative texture of the flap relative to the front. The cue can further be reflective and/or glow in the dark such that the user can access the free edge in poorly lit conditions.

For example, in the embodiment of FIG. 3A, the visual cue 90 is configured as a decorative pattern applied to the entirety of the flap 66. In one suitable embodiment, the pattern is formed by repeating pictorials. Of course, it should be understood that the pattern can be any combination of repeating pictorials, lines, shapes, characters, etc. In the embodiment of FIG. 3B, the visual cue 90 is configured as a color applied to the flap 66 that is different than the color of the front 64, meaning the colors have a different hue. One or more colors may also be different by virtue of having a different luminosity and/or saturation/vividness. Saturation/vividness is the intensity of the color from pale to dark. The elements may also have a different gloss/finish, from a matte finish, which tends to diffuse or scatter light, to a gloss finish, which is specular.

Referring to FIG. 8, hue is measured by the angular position around the circle 110. Two colors are considered different if they have first and second hues that are more than ±0.50 degrees from each other on the circle 110, alternatively±5 degrees, alternatively ±30 degrees, alternatively ±90 degrees, alternatively ±150 degrees and alternatively ±175 degrees. Value (luminosity) is measured along the Z-axis 112 between white and black. Colors are considered different if they have a value difference of at least 1% of maximum (Polaroid white reference standard). A value of one equates to white, while a value of 0 equates to black. Saturation/vividness is measured along the length of the radius (r). Colors are considered different if they have a saturation difference of at least 2.5% of maximum.

The hue, luminosity and saturation/vividness are measured as follows using the following equipment calibrated in the following way.

Equipment

Quantitative calorimetric measurements are typically made using a colorimeter or spectrophotometer. However, these instruments typically have large apertures (˜1 cm) requiring a large color block for meaningful determination, making them unsuitable for color determination of graphics that may be composed of narrow lines or points whose width is much less than the instrument aperture. Therefore, a Zeiss KS400 Image Analysis system was used for feature identification and calorimetric measurement.

The Zeiss KS400 used a Zeiss AxioCam color CCD camera (1300×1030 pixels, 3 channel color, 8 bit per channel) equipped with a 20 mm AF-Nikkor lens (f/2.8). The camera was mounted vertically facing down onto a sample stage and had an effective field of view was 97×80 mm. Incident sample stage illumination was by four incandescent floodlamps (Sylvania) on a double Variac (70%; 90%), resulting in an illuminance of approximately 11,000 lux. The lamps were above the left and right edges of the sample stage directed towards the field of view at approximately 45 degrees.

Calibration

The camera black reference was with the lens cap on. The camera white reference was a Polaroid 803 positive with 15 ms exposure. To account for the warm color illumination bias of the floodlamps, the red, green, and blue (RGB) values were offset using the white selection tool in the KS400 software, resulting in corrected RGB values that yielded a white image.

Sample Setup and Image Acquisition

Samples are placed on the stage (normal viewing angle) and under ¼″ plate glass to minimize topographical effects. Images of the color-bearing graphical portion are acquired at 15 ms exposure.

Image Analysis

Image analysis is performed in Matlab (v.6.5.1, release 13; Mathworks, Inc) with the Image Processing Toolbox (v4.0). RGB images were converted to hue, saturation, and value (HSV) space using Matlab's hsv2rgb.m command. Choosing a saturation lower limit of 0.05 (0-1 scale) resulted in practical detection of all the colored/inked portions of the graphic. The hue, saturation, and value (i.e. luminosity) densiometric distributions were calculated for the detected regions in each image.

Referring to FIG. 3C, the visual cue 90 includes a pattern formed over a portion of the flap, and an arrow shaped graphic 94 that is pointed at the edge 54. The graphic 94 defines a visual cue region 96, which can include a pattern or be of a different color than the remainder of the flap so as to further direct the user's attention to the edge 54.

Referring to FIG. 3E, the front is configured with a pattern 98, shown as alternating and spaced apart thin and thick vertical lines, that underlies the flap 66. In this embodiment, the flap 66 itself is the visual cue, with its plain configuration contrasted with the pattern 98 of the front portion. The flap 66 can be configured with any color, including white. Referring to FIG. 3F, the flap is configured with a first pattern 100 defining the visual cue, and the front is configured with a pattern 98 that is different from the first pattern 100.

In other embodiments (not shown), the visual cue could include a colored or patterned strip or wave formed on one or both of the flap and front. The visual cue could further include additional, separate pieces of material applied to one or both of the flap and front. In addition, the second portion of the front and the flap could be made identical (same pattern and/or color), but with the first portion of the front being a different color or pattern that is visible through the flap so as to provide a visual cue. For example, in one embodiment, the visual cue is configured by way of a colored adhesive, which is used to adhere the flap to the front. The adhesive is visible through the flap, which may be relatively see-through.

In another embodiment, the front and flap could have the same color and/or pattern, but with the flap being at least partly see-through such that the combined thickness of the first portion and portion of the flap overlying the first portion has a different appearance than the second portion. For example, the combined thickness of the portion of the flap overlying the first portion and the first portion may appear darker than the second portion of the front, thereby providing a visual cue directed at the free edge.

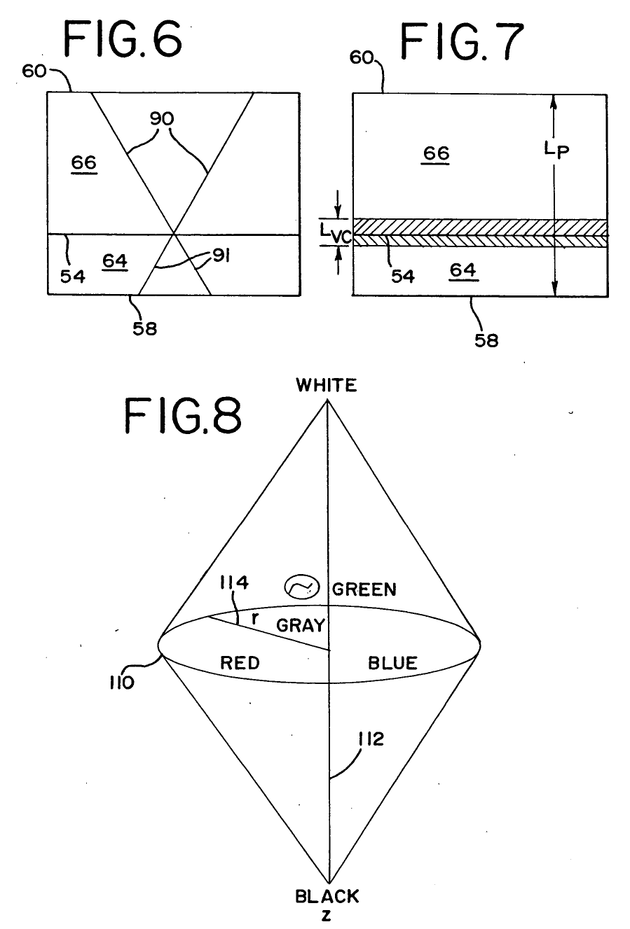

Referring to FIG. 6, both the flap 66 has a first visual cue 90 and the panel portion 64 has a second visual cue 91. Both of the first and second visual cues 90, 91 are directed and terminate at the free edge 54.

In yet another embodiment, shown in FIGS. 5A and 5B, a packaging component web 71 of material is provided with a color gradient, or fades, in a cross-direction 79 from a first side 75, where the material is darker (D) to a second side 73, where the material is lighter (L). As the web of material 71 is moved in the machine direction 77, a folded product component, e.g., a tri-folded feminine pad, is applied to the web of material 71. The web 71 is then folded around the product component, with the first side 75 forming the exterior free edge 54 that overlies the front and is spaced from the second side 73, which forms the interior free edge 56. Alternatively, the light side forms the exterior free edge, with the dark side forming the interior free edge. Cross-direction 79 side seals are then successively applied to the folded web, and the web is then cut at the side seals to form a plurality of discrete personal care products.

The color gradient provides a processing advantage in that the contrast between the dark and light ends 75, 73 provides a visual cue, but without concern about where the fold line 60 is formed. In this way, the color gradient provides a tolerance for forming the fold line 60, in contrast with embodiments where a color or pattern ends at the fold line.

In another embodiment, the flap 66 and the front 64 are each of one color, which when overlapped form a third color. The visual cue can include a line or intermittent pattern disposed on the flap or front adjacent the free edge. A pattern forming the visual cue can extend along a portion of the length of the flap or front, along the entirety thereof, and can vary across the width thereof. Of course, it should be understood that the visual cue can take many forms, and can include various combinations of colors, patterns, shading etc.

In some embodiments, the sensory cue includes one or both of a visual cue and a tactile cue. For example, the flap can have a different texture than the underlying panel, so as to form a tactile cue. In some embodiments, the tactile cue is formed by an embossment, or different embossments. Similarly, a separate piece of material applied to one or both of the flap or front, may have a different texture or feel than the other thereof, thereby providing a tactile cue as to the location of the free edge.

In one embodiment, the visual cue is located on the front and is directed at the free edge so as to provide indicia to the user about where their hand should be located such that it can be inserted to lift the flap. In another embodiment, the visual cue is located on the flap opposite where the hand should be located. In either case, the visual cue provides indicia of the location of the free edge, but also indicia of where the user's hand should be located.

In an alternative embodiment, shown in FIG. 7, the visual cue 90 is disposed on both sides of the free edge 54, and does not terminate at the free edge. Rather, in this embodiment, the visual cue terminates on each side of the free edge. In one embodiment, the length of the visual cue (Lvc) is less than about 33% of the overall length (Lp) of the product, with the free edge 90 disposed within the coverage of the visual cue. In other embodiments, Lvc is less than about 25% of Lp, and more desirably less than about 10% of Lp.

Referring to FIGS. 1 and 3A-3F, in operation, the user locates the free edge 54 of the packaging component 50 using the sensory cue (visual or tactile) cue 90. The user then grasps the free edge 54 or flap 66 and opens the packaging component to access the personal care product component. The user can then remove the product component and discard the packaging component.

Although the present invention has been described with reference to various embodiments, those skilled in the art will recognize that changes may be made in form and detail without departing from the spirit and scope of the invention. As such, it is intended that the foregoing detailed description be regarded as illustrative rather than limiting and that it is the appended claims, including all equivalents thereof, which are intended to define the scope of the invention.

1. A personal care product comprising:
a packaging component comprising a panel and a flap having a free edge positioned adjacent said panel, wherein at least one of said flap and said panel comprises a sensory cue directed at said free edge of said flap, wherein said sensory cue provides indicia as to the location of the free edge and wherein said sensory cue terminates at said free edge; and
a personal care product component disposed in said packaging component.

2. The personal care product of claim 1 wherein said panel comprises a front panel, and wherein said packaging component further comprises a back panel, wherein said flap is connected to said back panel and wherein said free edge overlies at least a portion of said front panel.

3 The personal care product of claim 1 wherein said product component comprises an absorbent product component.

4. The personal care product of claim 1 wherein said flap comprises said sensory cue.

5. The personal care product of claim 1 wherein said panel comprises said sensory cue.

6. The personal care product of claim 1 wherein said sensory cue comprises a visual cue.

7. The personal care product of claim 6 wherein said visual cue comprises a first color, wherein said other of said panel and said flap has a second color different than said first color.

8. The personal care product of claim 6 wherein said visual cue comprises a pattern printed on said one of said flap and said panel.

9. The personal care product of claim 6 wherein said visual cue comprises an arrow shape directed at said free edge.

10. The personal care product of claim 6 wherein said visual cue comprises a first pattern formed on one of said flap and said panel and wherein said other of said flap and said panel comprises a second pattern different from said first pattern.

11. The personal care product of claim 1 wherein said sensory cue comprises a tactile cue.

12. A personal care product comprising:
a packaging component comprising a front panel, a back panel and a flap connected to said back panel and having a free edge overlying at least a portion of said front panel, wherein one of said flap and said front panel comprises a visual cue directed at said free edge of said flap, wherein said visual cue provides indicia as to the location of the free edge and wherein said visual cue comprises a pattern formed on one of said flap and said front panel; and
a personal care product component disposed in said packaging component.

13. The personal care product of claim 12 wherein said product component comprises an absorbent product component.

14. The personal care product of claim 12 wherein said flap comprises said visual cue.

15. The personal care product of claim 12 wherein said front panel comprises said visual cue.

16. The personal care product of claim 12 wherein said visual cue further comprises an arrow shape directed at said free edge.

17. The personal care product of claim 12 wherein said pattern comprises a first pattern, and wherein said other of said flap and said front panel comprises a second pattern different from said first pattern.

18. A method of using a personal care product comprising:
providing a personal care product component disposed in a packaging component comprising a front panel, a back panel and a flap connected to said back panel and having a free edge overlying at least a portion of said front panel, wherein one of said flap and said front panel comprises a visual cue directed at said free edge of said flap, wherein said visual cue terminates at said free edge;
locating said free edge using said visual cue; and
grasping said flap and opening said packaging component to access said personal care product component.

19. The method of claim 18 wherein said product component comprises an absorbent product component.

20. The method of claim 18 wherein said flap comprises said visual cue.

21. The method of claim 18 wherein said front panel comprises said visual cue.

22. The method of claim 18 wherein said visual cue comprises a first color, wherein said other of said front panel and said flap have a second color different than said first color.

22. The method of claim 18 wherein said visual cue comprises a pattern printed on said one of said flap and said front panel.

23. The method of claim 18 wherein said visual cue comprises an arrow shape directed at said free edge.

24. The method of claim 18 wherein said visual cue comprises a first pattern formed on one of said flap and said front panel and wherein said other of said flap and said front panel comprises a second pattern different from said first pattern.

25. A personal care product comprising:
a packaging component having a first length and comprising a panel and a flap having a free edge positioned adjacent said panel, wherein said flap and said panel comprise a sensory cue disposed across said free edge of said flap, wherein said sensory cue provides indicia as to the location of said free edge, and wherein said sensory cue has a second length less than about 33% of said first length; and
a personal care product component disposed in said packaging component.

26. The personal care product of claim 25 wherein said second length is less than about 25% of said first length.

27. The personal care product of claim 26 wherein said second length is less than about 10% of said first length.
